(19) United States
(12) Patent Application Publication
(10) Pub. No.: US 2021/0241890 A1
(43) Pub. Date: Aug. 5, 2021

(54) OPTIMIZED CLIENT-STAFF COMMUNICATION SYSTEMS DEVICES AND METHODS FORDIGITAL HEALTH FACILITY

(71) Applicant: HILMA LTD., Jerusalem (IL)

(72) Inventors: Yosef TSURIA, Jerusalem (IL); Arnold ZUCKER, Hashmonaim (IL); Michal OPHIR, Jerusalem (IL)

(21) Appl. No.: 17/163,614

(22) Filed: Feb. 1, 2021

Related U.S. Application Data
(60) Provisional application No. 62/967,599, filed on Jan. 30, 2020.

(51) Int. Cl. G16H 40/20; G06Q 10/06; G06N 5/02

(52) U.S. Cl. CPC G16H 40/20; G06N 5/025; G06Q 10/063116

(57) ABSTRACT

There are provided methods and systems for managing service requests of patients and staff in a hospital and generating a matching dynamic interface, the method comprising: receiving or obtaining via the network from the patient' devices service requests; receiving or obtaining, patient inputs and general inputs from one or more resources; receiving or obtaining staff inputs; analyzing the received and obtained service requests, data inputs, staff inputs and general inputs using one or more analyzer modules to yield analysis results; categorizing the analysis results to groups categories and generating for each of the categorized groups the matching dynamic interface based on the analysis results.

### CROSS-REFERENCE TO RELATED APPLICATIONS

The present application claims the benefit of U.S. Provisional Application Ser. No. 62/967,599 filed on Jan. 30, 2020, entitled “Client-Staff Communication System in Digital Health Facility” (attorney docket no. HL001/USP) which is incorporated herein by reference in its entirety.

### TECHNICAL FIELD

The present disclosure relates to client-staff communication systems devices and methods for Digital Health Facilities, more specifically, but not exclusively, to automated dynamic and optimized healthcare communication systems devices and methods for health facilities including patients (or clients) and medical staff on site, such as hospitals.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

Hospital ‘Call’ systems known also as nurse-call-systems for allowing patients to summon help from the medical staff have been in existence for a long time in-patient healthcare facilities such as hospitals.

Some more advanced nurse call systems include speakers and microphones. In operation, a patient may initiate a nurse call by pressing a nurse call button located for example on the patient's bed or on a hand-held unit. Once the patient places a nurse call, a caregiver may answer the call using the microphone and speakers located in his station and may speak with the patient which uses the microphone and speakers located in his room.

To facilitate such voice communications a microphone and speaker are located somewhere in the patient room. Oftentimes the microphone and speaker are included in the pillow speaker unit or in an audio station mounted to a wall of the patient room or to a headwall unit which, in turn, is mounted to a wall of the patient room.

Other known call systems include in-ward signaling systems such as RED call buttons. However, these systems are ineffectual as these systems do not differentiate types of calls such as directing calls to nurses or physicians according to the type of call and therefore priorities are hard to establish and efficiency in care suffers. A system that can help caregivers distinguish between time-sensitive or critical needs and less urgent requests can help both patient and caregiver better manage their time in the hospital.

However, these solutions have not advanced much beyond the basic bell or call button. Moreover, current systems are extremely inefficient to the point of frustrating patients, families, and staff, and sometimes even jeopardizing the care and health of the patient.

Moreover, these monitoring systems may also provide wrong detections for non-standard positions of the passengers such as in cases where one or more passengers are leaning outside the seat position or when body parts invade neighboring seats.

Furthermore, prior monitoring systems require significant and costly processing resources and typically the needed processing time for training the monitoring systems and the processing units are long resulting in resource dissipation.

In light of the above, improved healthcare communication systems, devices, and methods for helping the medical staff such as caregivers distinguish between time-sensitive or critical needs and less urgent requests and which can help both patients, staff, and caregivers better manage their time in the hospital and further overcomes at least some of the above mentioned deficiencies of the prior detection would be beneficial. Ideally, such systems would be compact, integrated with other devices and systems such as the hospital systems and units and the users (e.g. patients, staff caregivers) devices such as the users' mobile phones, sufficiently rugged, low in cost practical, and convenient to use for both patients staff and caregivers.

### SUMMARY OF THE INVENTION

According to a first aspect of some embodiments there is provided a method for managing service requests of patients and staff in a hospital and generating, respectively, for each of said patients and staff a matching dynamic interface, the method comprising: receiving or obtaining via the network from the patient' devices service requests; receiving or obtaining, patient inputs and general inputs from one or more resources; receiving or obtaining staff inputs respectively from the staff or from the one or more resources; analyzing the received and obtained service requests, data inputs, staff inputs and general inputs using one or more analyzer modules to yield analysis results; categorizing the analysis results to groups categories wherein said categorized groups comprise staff groups, patient groups and hospital database; generating for each of said categorized groups the matching dynamic interface based on the analysis results, wherein the matching dynamic interface comprises output data which match the group category medical needs and whom the analysis results are intended.

In an embodiment, the analysis comprises: analyzing the received and obtained service requests and data inputs using a patient analyzer module, and identifying types of service requests in said obtained service requests that are both available and relevant for a particular patient of said patients at a particular time; displaying said identified/selected service requests at said matching dynamic interface.

In an embodiment, the analysis comprises: analyzing the received and obtained service requests and data inputs using an Auto-Request Services Generator Module; automatically or autonomously generating system-initiated service requests for the patients.

In an embodiment, the system-initiated service requests for the patients are generated using one or more specific rules or using Artificial Intelligence (AI) methods to recognize and flag situations.

In an embodiment, the AI methods are Anomaly Detection methods.

In an embodiment, the analysis comprises: analyzing the received and collected general inputs and staff inputs or data received from general data and management systems using a staff analyzer module to yield staff data.

In an embodiment, the staff data comprises personalized tasks list specifically for each staff category.

In an embodiment, the method comprising: assigning service requests to the appropriate staff.

In an embodiment, the method comprising: prioritizing tasks in the personalized tasks list.

In an embodiment, the method comprising automatically or autonomously optimizing the received and obtained requests and assigning service requests to the appropriate agent.

In an embodiment, the method comprising displaying the prioritized tasks on the staff interface.

In an embodiment, the method comprising generating a separate dynamic interface for the staff groups and for the patient groups.

In an embodiment, the method comprising analyzing the received and obtained collected general inputs, staff inputs or data received for example from general data and management system; predicting service request needs of the patients.

In an embodiment, the service request needs comprise one or more of: supply needs and shortages, seasonal personnel needs, hospital bed shortages.

In an embodiment, the service requests comprise any specific need of the patients, which can be fulfilled by the staff.

In an embodiment, the staff inputs are received or obtained via the network from the staff's devices.

In an embodiment, the matching dynamic interface is a dynamic graphical interface comprising the output data, and wherein said output data is configured and enabled to continually, automatically or autonomously being updated and changed according to the patients' needs, status and location.

In an embodiment, the output data comprises one or more of: graphical data, one or more icons.

According to a second aspect of some embodiments there is provided a system for managing service requests of patients and staff in a hospital and generating, respectively, for each of said patients and staff a matching and dynamic interface, the system comprising: a memory, which is configured to hold one or more of patient inputs or general inputs; and a processor, said processor is configured to: receive or obtain via the network from the patient' devices service requests; receive or obtain, the patient inputs and general inputs from the memory; receive or obtain staff inputs respectively from the staff or from the one or more resources; analyze the received and obtained service requests, data inputs, staff inputs and general inputs using one or more analyzer modules to yield analysis results; categorize the analysis results to groups categories wherein said categorized groups comprise staff groups, patient groups and hospital database; generate for each of said categorized groups the matching dynamic interface based on the analysis results, wherein the matching dynamic interface comprises output data which match the group category medical needs and whom the analysis results are intended.

According to a third aspect of some embodiments there is provided a computer software product, comprising a non-transitory computer-readable medium in which program instructions are stored, which instructions, when read by a computer, cause the computer to provide a method for managing service requests of patients and staff in a hospital and generating for each of said patients and staff a matching dynamic interface, the method comprising: receiving or obtaining via the network from the patient' devices service requests; receiving or obtaining, patient inputs and general inputs from one or more resources; receiving or obtaining staff inputs respectively from the staff or from the one or more resources; analyzing the received and obtained service requests, data inputs, staff inputs and general inputs using one or more analyzer modules to yield analysis results; categorizing the analysis results to groups categories wherein said categorized groups comprise staff groups, patient groups and hospital database; generating for each of said categorized groups the matching dynamic interface based on the analysis results, wherein the matching dynamic interface comprises output data which match the group category medical needs and whom the analysis results are intended.

### BRIEF DESCRIPTION OF THE DRAWINGS

A better understanding of the features and advantages of the present disclosure will be obtained by reference to the following detailed description that sets forth illustrative embodiments, in which the principles of embodiments of the present disclosure are utilized, and the accompanying drawings.

FIG. 1A shows a schematic diagram of a health communication system, in accordance with some embodiments of the present disclosure;

FIG. 1B shows a schematic diagram of a user device, in accordance with some embodiments of the present disclosure;

FIGS. 2A-2F show respective examples of related UI (user interface) display views, in accordance with some embodiments of the present disclosure;

FIG. 3A is a high-level schematic flowchart of a method for managing requests of users in hospitals and generating for each user a matching interaction tool to allow effective and optimized operation and control of the requests, in accordance with some embodiments of the present disclosure;

FIG. 3B shows a detailed flowchart of the method of FIG. 3A, in accordance with some embodiments of the present disclosure;

FIG. 4A shows a high-level block diagram of a system including a call analyzer module, in accordance with some embodiments of the present disclosure;

FIG. 4B is a block diagram illustrating in details an example of the modules included in the call analyzer module of FIG. 4A module, in accordance with some embodiments of the present disclosure;

FIG. 5 shows a flowchart of a method for identifying the types of service requests that are both available and relevant for a particular patient for example at a particular time, in accordance with embodiments;

FIG. 6 shows a flowchart of a method for automatically generating service requests that are both available and relevant for a particular patient for example at a particular time, in accordance with embodiments; and

FIG. 7 shows a flowchart of a method for automatically optimizing received and/or collected requests including assigning service requests to the appropriate agent, and to prioritizing the tasks of each agent, in accordance with embodiments.

### DETAILED DESCRIPTION OF THE INVENTION

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore, the invention is not limited by that which is illustrated in the figure and described in the specification, but only as indicated in the accompanying claims, with the proper scope determined only by the broadest interpretation of said claims.

The present disclosure relates to client-staff communication systems devices and methods for Digital Health Facilities, more specifically, but not exclusively, to automated, dynamic and optimized healthcare communication systems devices and methods for health facilities including patients (or clients) and medical staff on site, such as hospitals.

Prior to the detailed specification of the invention being set forth, it may be helpful to set forth definitions of certain terms that will be used hereinafter.

As used herein, like characters refer to like elements.

The term ‘Hospital’ or ‘health facility’ or ‘medical center’ as used herein are defined as locations or institutions to signify any type of care facility that has patients (or clients or users) and staff on site, such as physician or agents. Typically, hospitals and health facilities include Digital Health Facilities or systems.

The term ‘Patient(s)’ as used herein is defined as individual(s) who require/benefit from the services in the system (e.g. system 100 or modules such as call analyzer module 400) or app.

The term ‘Service Request(s)’ as used herein is defined as any specific need of the patient, which can be fulfilled by an agent or staff. A service is either expressly requested by the patient or deduced by the system. Examples of patient-initiated service requests include requests for toilet/shower help, change of bedding, assistance with pain, meal service issues, etc. Examples of system-initiated service requests include scheduled infusions and regular meal services as well as a nurse check up on a patient who has NOT asked for a bedpan all day, or who's oxygenation level (measured by sensors) is too low.

The term ‘agent(s)’ or ‘staff’ as used herein are defined as hospital personnel who come in direct contact with patients and are equipped to fulfill service requests. Agent(s) may include nurses, orderlies, physicians, technicians, social workers, etc.

The configurations disclosed herein can be combined in one or more of many ways to provide management and optimization of hospitals (or any healthcare facilities or institutions) resources such as the hospitals' digital resources including for example client-staff communications systems for the benefit of the hospital's patients and staff (doctors, nurses, hospital orderly) and administration.

In accordance with embodiments, the systems devices and methods are configured and enabled to manage, for example automatically and/or autonomously the auxiliary needs of hospital clientele such as the hospital patients, staff and administration.

The systems, devices and methods include, in accordance with embodiments managing service requests of patients and staff in a hospital and generating, respectively, for each of the patients and staff a matching dynamic interface and collecting, understanding and prioritizing data which includes patient needs during, before and after hospitalization, with a focus on the complete range of services a hospital provides, rather than on the medical treatment alone.

In accordance with embodiments, the systems devices and methods are configured and enabled to provide separate applications for use by patients, care providers (agents), and administrators. For example, the graphical user interface and data provided to a patient, in accordance with embodiments, differs from the graphical user interface and data provided to care providers (agents), and administrators.

In accordance with embodiments, there are provided devices, systems and methods for receiving data such as patient's or staff data and analyzing the received data to yield analysis results which include one or more tasks/requests and further differentiate and/or prioritize the tasks based on the type of help required and urgency of each task. In an embodiment, the devices, systems and methods may include automatically and/or autonomously signaling the appropriate support agent to deal with specific tasks/requests and supply sufficient information to allow the agent(s) to organize and prioritize their responses.

The system and devices, in accordance with embodiments, may be connected to and/or be in communication with one or more sensors such as sensors at the patient and/or patient's bed and/or to hospital IT systems for access to data and/or to external or internal resources in the hospital or outside the hospital. For example, as an app on the patient's phone the system may have access to some of the phone's sensors or other IoT (Internet of Things) devices.

In some cases, the systems and devices may not be allowed a direct access to the hospital's patient data system and therefore a demilitarized zone (DMZ), e.g. a safe repository of data that has been sent from the hospital IT system but cannot be directly accessed by external systems, may be used.

In some embodiments, some data could reside in the DMZ anonymously, using a temporary hospital ID (or bed designation) for local tracking. Significant events would be reported back from the DMZ to the hospital IT system, while maintaining anonymity in the DMZ.

In accordance with embodiments, there are provided systems and methods configured and enabled to evaluate ward performance based on number, severity and time for responses.

In some cases, the UI and tasks list or data may include messages that can also be sent to hospital non-medical departments like maintenance for fixing air conditioning, bathrooms, etc.

In some cases, the modules such as module 400 is configured and enabled to categorize (e.g. divide) patients and optionally assign rooms on the basis of specific qualities or needs, e.g. (obviously male-female), but also large vs. small patients, loud-quiet, much support-little support required, language preferences, gluten intolerance, patients need to be in another department at specific times, X Rays, MRI, etc. This can be gleaned from patients records or updated as the nurses get to know a new patient, or based on patient interviews.

In some embodiments, the system and methods are configured and enabled to evaluate requests from patients on a priority basis taking into account time sensitivity, severity, availability, etc. It allows a form of triage for all non-emergency patients allowing a dynamic re-evaluation at each request, beyond the ‘red’ emergency button.

In some embodiments, the system and methods are configured and enabled to monitor and backup tablet at the entrance to each room. The monitors will display a patient's status and location.

In some embodiments, the system and methods are configured and enabled to track (via sensors, data entry, or written note) where the patient is, e.g. taken for x-ray or MRI, sent to a different department, to OR, etc.

In some cases, the received data may include data received from patients and/or data received from the medical staff and/or data received from the user devices (e.g. portable devices such as the patient's mobile phone) and/or from sensors such as GPS.

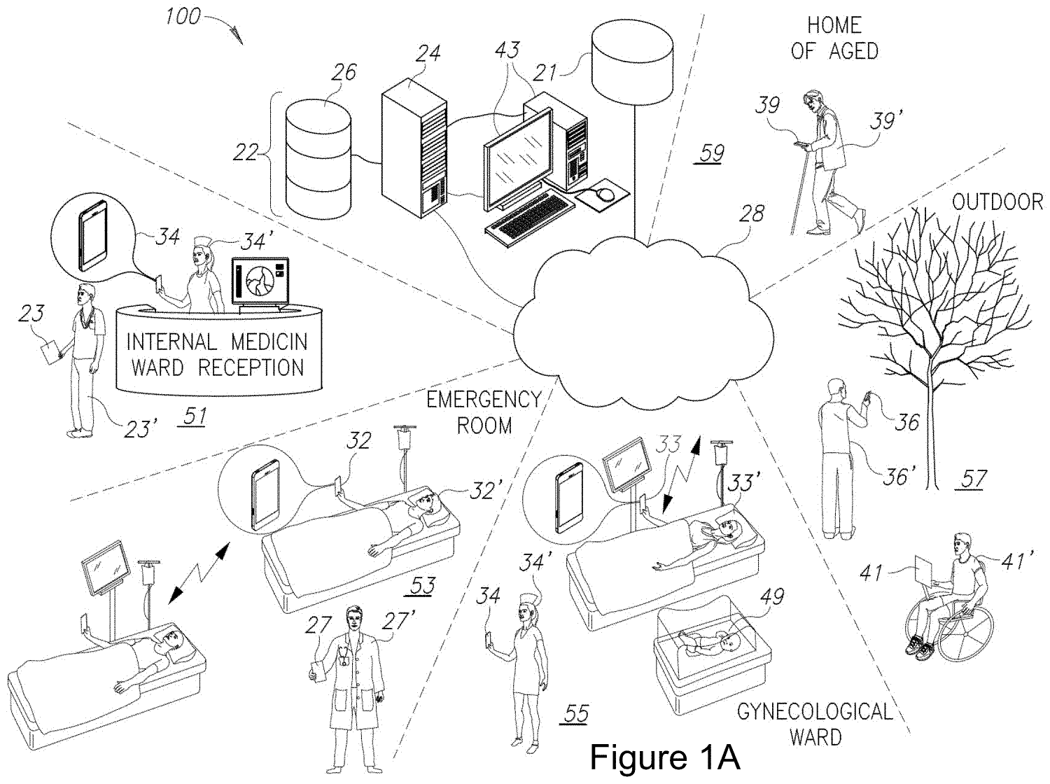

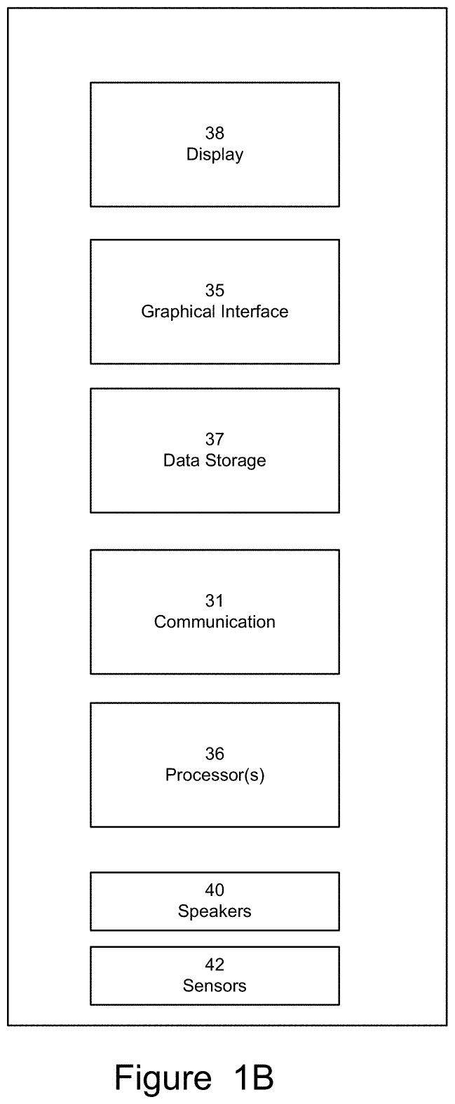

FIG. 1A is a schematic diagram of a health communication system 100, in accordance with embodiments. A server such as a hospital server 22 comprises one or more processors 24 and a memory 26 (e.g. data storage) which may store one or more of data assets such as medical data assets of patients and general info (e.g. general inputs) such as number of beds in the hospital, supply status, general statistics and the like. More specifically, memory 26 may store for example patients' data, such as personal health data, number of patients, number of occupied hospital beds in each department, supply status at each of the hospital's departments (e.g. bedding, medical equipment etc.) and/or any data the one or more processors 24 received from user clients (patients, nurses, staff etc.) and/or from a system central data storage 21. Typically, server 22 comprises a suitable general-purpose computer (or a cluster of such computers), which has been programmed in software to carry out the functions that are described herein. This software may be downloaded to the computer in electronic form, over a network. Additionally or alternatively, the software may be stored on tangible, non-transitory computer-readable media, such as magnetic, optical, or electronic memory media.

Server 22 communicates over a network 28 with multiple client devices 23, 27, 32, 33 34, 36 39, and 41 of respectively the hospital clientele such as nurse 34′, doctors 27′ sanitarian 23′ patients 32′, 39′ mother 33′, baby 49, and agents. Typically, network 28 comprises the public Internet, and server 22 communicates with the client devices via a suitable Web interface, as is known in the art. In some cases, a remote processing and control unit can be coupled to the client's hand held device (e.g. a cell phone). The remote processing and control unit can be a cloud based system which can transmit analyzed data or results to a user. The association can be through a physical connection or wireless communication, for example.

In some embodiments, the devices are connected to the communication network that allows users to share the information obtained. An updatable database located in the “cloud” (i.e. the distributed network) constantly receives the analysis results for each individual users and updates itself in real time, thus enabling each data analysis to be made with greater accuracy and confidence.

Alternatively, the server and clients may communicate using any other suitable sort of network and interface.

Client devices 23, 27, 32, 33, 34, 36 39 and 41 may comprise, for example, desktop, laptop, or tablet computers, media consoles, personal digital assistants or smartphones, or any other sort of device with the types of network, video and audio interfaces and computing capabilities needed to interact with server 22.

By way of example, as illustrated in FIG. 1B, each or some of the client device 23, 27, 32, 33, 34, 36 39 and 41 may comprise or may be a computer or a portable device such as a smartphone with processor(s) 36, data storage 37, a video display 38 and speakers 40 for playing media assets, along with one or more sensors 42 such as video camera, microphone for recording, communication circuitry 31 for transmitting/receiving data from external devices or systems. Some of the Client devices may be differently equipped, some may be similarly equipped though in different configurations.

As shown in FIG. 1A patient's location and condition changes, as do his/her needs relative to that change. The needs of a patient can be very different depending on whether the patient is in bed, or whether the patient such as patient 36 is outdoor 57 or in the cafeteria, coming out of x-ray or rehab, and whether he is on the emergency room 53, or the patient is a mother at the gynecological ward 55 or a nurse 34′ or sanitarian 23′ at an internal medicine ward reception 51.

Even in the same location, time can be a major factor, e.g. when a patient is waking up after surgery he has a different set of needs and should have a different set of icons then the person in the neighboring bed who is already a week into post-operative recuperation.

In accordance with embodiments, these changes may be evaluated for example using one or more of the systems and methods modules (e.g. patient analyzer module 402), which allows the system to take into account such changes.

The location also has different assets and capabilities depending on time. A night shift or weekend or holiday staffing might have a smaller staff allocation and therefore certain needs will not be filled by the staff, like shower help when staff is minimal, etc. Rehabilitation equipment might be unavailable, or transportation options may be changed, and these changes need to be reflected in what the patient can choose; hence the methods and systems are further configured using for example the staff analyzer module 412 to collect and evaluate all the relevant changes to staff and site.

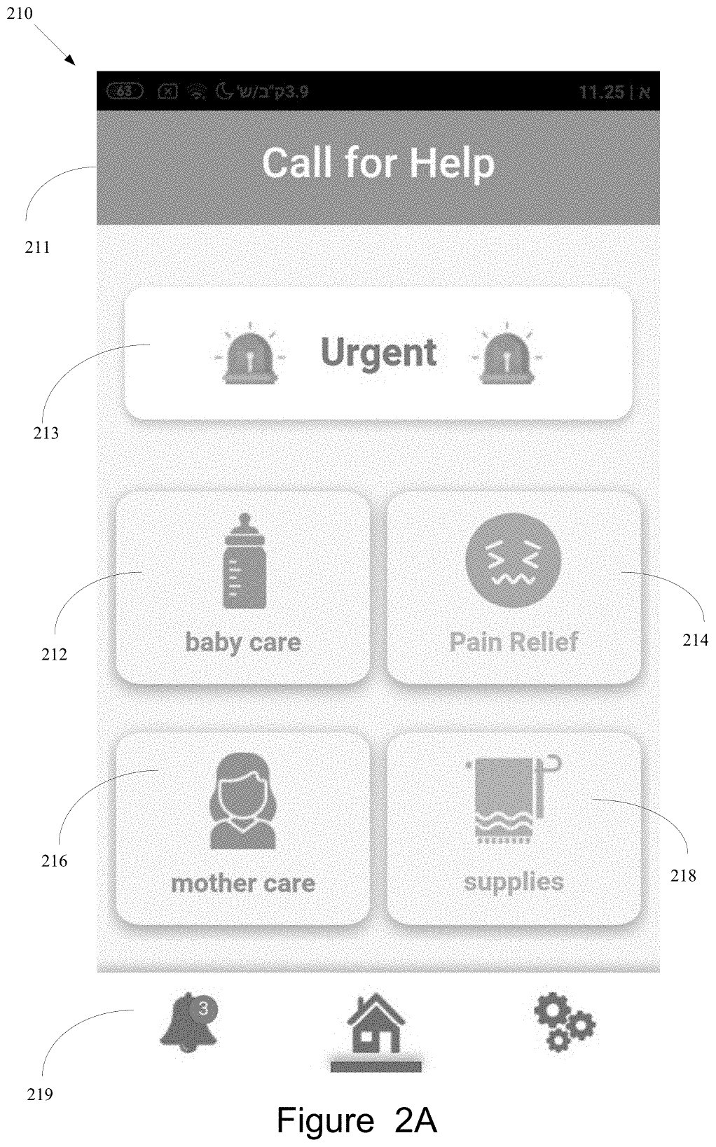

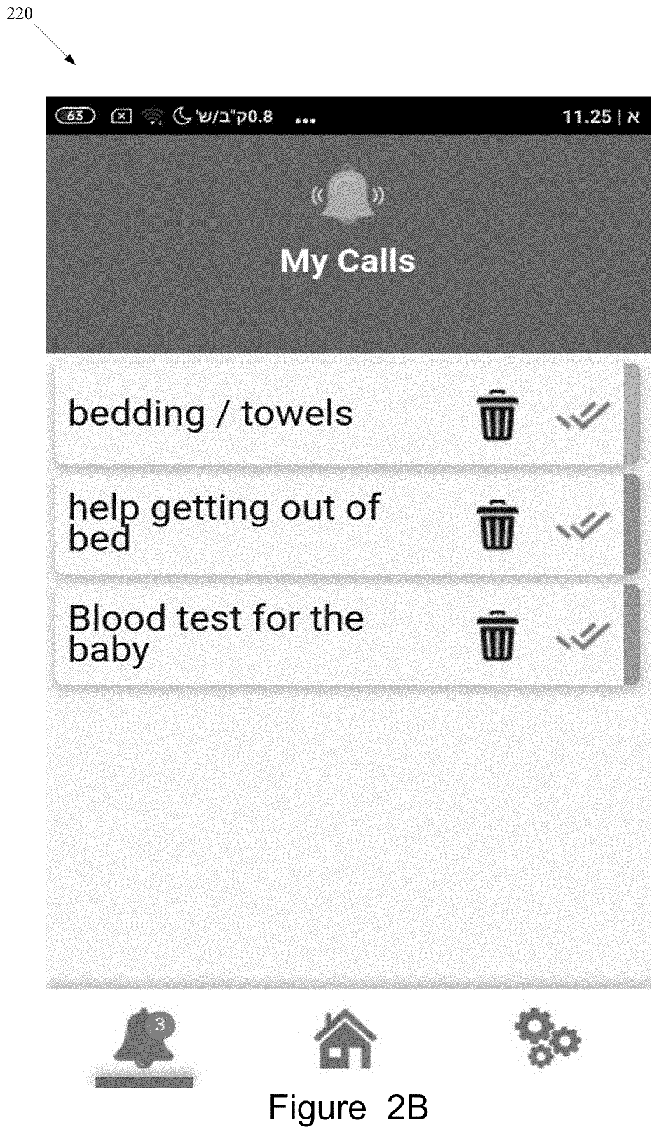

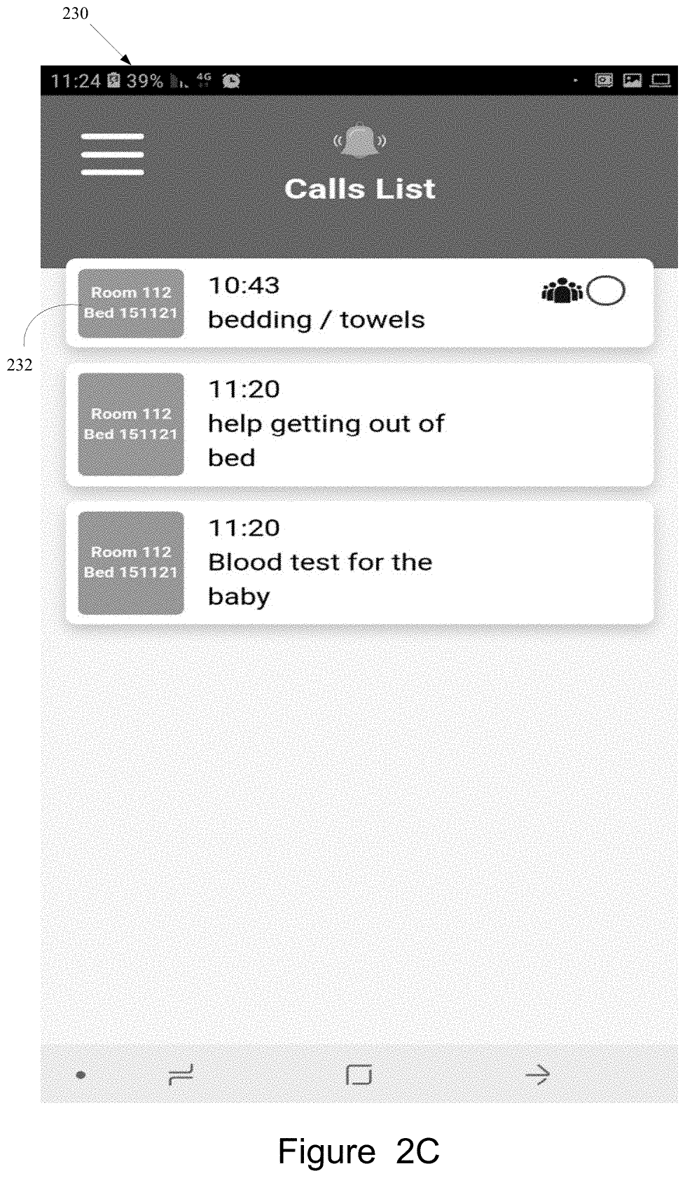

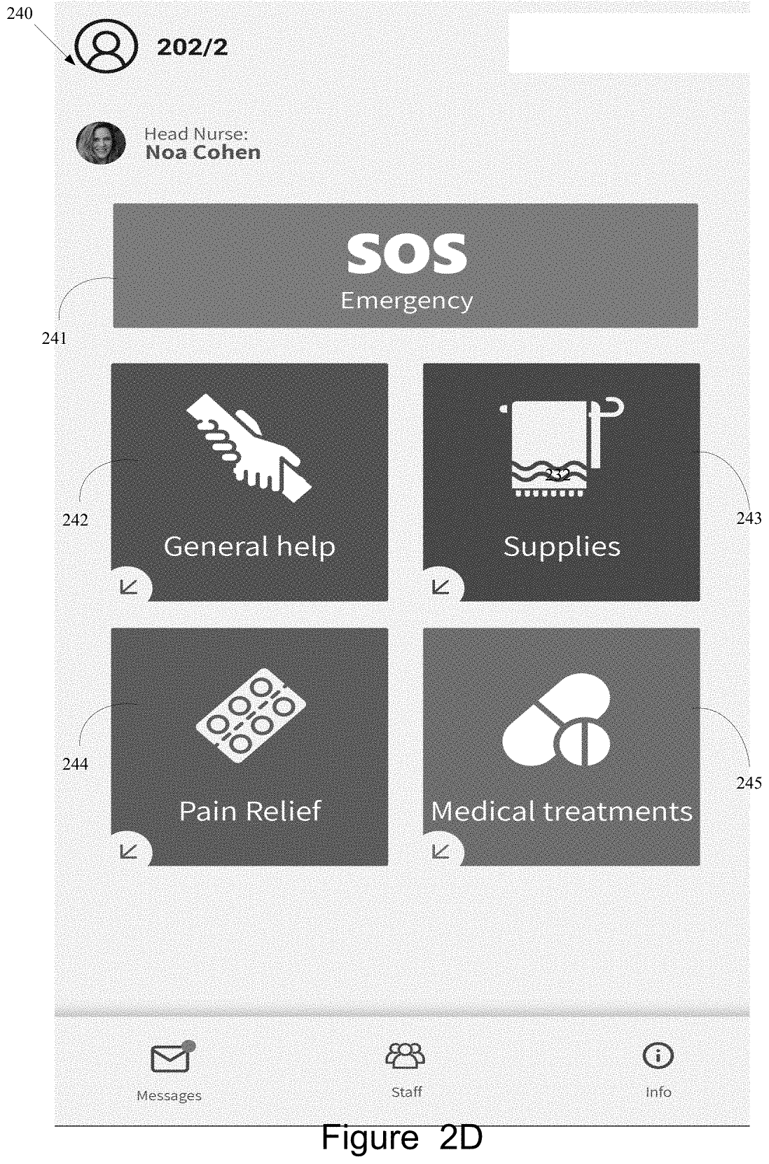

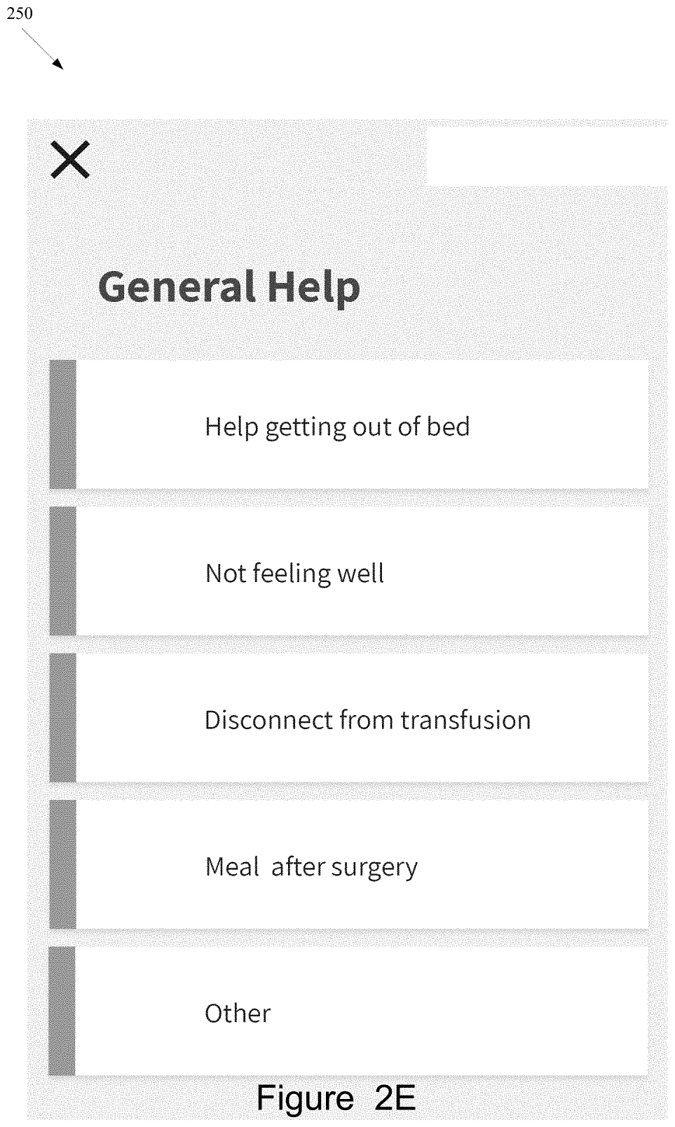

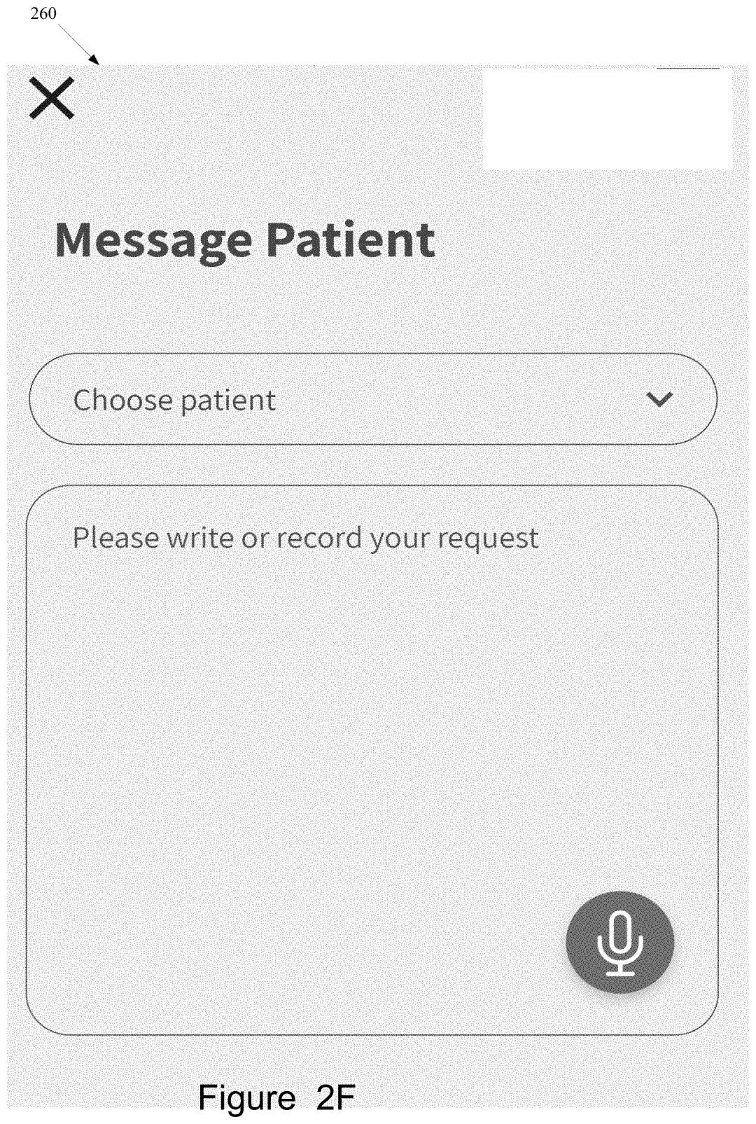

When staffing is changed or the patient's condition has changed the icons, which to a significant degree express support availability or priorities, may change as well as illustrated in FIGS. 2A-2F. A postoperative patient might have various eating options removed from their interface and only emergency or high priority call icons shown.

This location-situation-time sensitivity applies not just to the menu of icons that may be changed but also to the specific triggers that will change. Context-awareness is evaluated for relevance and appropriate system response by the call analyzer module 400 and will be reflected in icon and trigger changes.

Context specificity of icons and triggers applies to situations even where the patient is no longer in hospital for example outdoor 57 or at the home of the aged 59. While a patient can push the emergency button when in a hospital bed and get a particular response, like a nurse coming to her aid, that will be different when triggered at home where it might trigger an ambulance or other first responder service.

This same system, in accordance with embodiments, will be taken as an app to the patients' homes to continue the relationship to the hospital or to a 3rd medical care system. Upon exiting the hospital, for example, outdoor 57 or at a home of the aged 59 the specific icons may change but, in some cases, the general layout would continue much the same. For example, where activity allows for a similar usage, e.g. a red emergency button in the hospital would now be configured to call an ambulance, the module would reconfigure that part of the interface. Doctors (and caregivers) messages might be moved to a hospital (or 3rd party) system for secure access by all involved. Interaction with the hospital will allow for efficient scheduling of services at the hospital if required, like MRIs and x-rays, etc., because it allows the hospital to know who is coming from outside and what their priority is.

According to some embodiments, the systems and methods can also be tied to a 3rd party that dispenses services and aids. A nurse or doctor can help the patient decide what appliances or services may be of use for the patient being discharged, e.g. walker, wheelchair, ventilator, etc., and have the 3rd party manage the delivery and use in the normal manner.

Out of hospital the systems and methods are configured and enabled to be tied to local sensors or devices, via Bluetooth or Wi-Fi for example, or allow for data entry of such data read from sensors or devices at the patients' home (or facility) like thermometers—temperature, blood sugars, heart rate, blood pressure, etc.

In some embodiments, the system and methods may be in communications with an effective messaging and response platform from medical and supervisory staff, like Amigo, to allow for remote patient care.

Therefore, the interface (e.g. graphical interface 35) provided in accordance with embodiments for each user (e.g. client device) may be a dynamic graphical interface and may comprise one or more icons which automatically and/or autonomously and/or continually updated and changed according to the user's (e.g. patient or staff) needs, status (e.g. medical status) and/or location and/or time. Utilizing a dynamic graphical interface on the patient's device such as his phone enables the patient to signal to the medical staff (e.g. nurses and/or doctors) exactly what is needed and/or what he needs, e.g. pain medication vs. a cup of tea. Respectively, a dynamic graphical interface on the staff device enables the staff to signal to one another or to the patient. The system 100 is designed such that the graphical interface, which may reside on a dedicated device or alternatively as an app on a smartphone (or similar device, e.g. tablet, etc.) will display for example a set of icons, where each icon triggers, for example, a different help request.

Hence, the interface of mobile device 33 of mother 33′ at the gynecological ward will include information and graphical interface as illustrated in FIG. 2A and FIG. 2B while the interface of the mobile device 32 of patient 32′ at the emergency room different will include information and graphical interface as illustrated in FIGS. 2D-2F. Accordingly, the interface of mobile device 34 of nurse 34′ at the gynecological ward will include information and graphical interface as illustrated in FIG. 2C while the interface of the mobile device 27 of doctor 27′ at the emergency room 53 will include different information and graphical interface.

It is noted that other interfaces may be used such as textual and/or voice and/or VR (Virtual Reality) and/or AR (Augmented Reality) may be used for example by using dedicated devices such as VR or AR goggles etc.

In accordance with embodiments, as illustrated in FIGS. 2A-2F, the graphical interfaces would include easily understood representations of likely patient needs based on for example his or her medical conditions and/or medical needs and ward he or she is currently hospitalized. For example, the graphical interface may include information such as bathroom/shower access, food requests, and medication requests, including a standard ruler for pain measures to inform nurses of severity and staff tasks or specific requests (e.g. water) and in some cases also the severity of the patient pain (e.g. Pain level 5).

For example, in accordance with some embodiments, an icon may trigger a call to some assisting person, such as a ward volunteer, for providing a cup of water, while another icon would trigger a high priority call to a nurse or attending physician. An icon or set of icons can trigger a message assigned routing directly to an appropriate destination using defined connections, such that nurses calls can be routed to a head nurses station display, while calls to other volunteers can be pre-directed to another display or to the nurses display or management station in order to be assigned by a staff person.

FIG. 2A, FIG. 2B and FIG. 2C show, respectively, examples of screenshots of a mother and nurse interface 210, 220 and 230 which automatically match a mother's needs at the hospital's gynecological ward, in accordance with embodiment. In the example shown in FIG. 2A the graphical interface 210 includes a display that is tailored according to the needs of a mother who just gave birth and accordingly her graphical interface will include icons that match the mother immediate needs while she is for example at the maternity ward or gynecological ward. In some cases, the graphical interface includes a ‘Call for Help’ window 211 including one or more of the following icons which exactly match the specific needs of the mother: ‘urgent’ 213—for urgent matters, ‘baby care’ 212—for receiving help from a nurse with the newborn, ‘Pain Relief’ 214, ‘mother care’ 216, supplies 218—for receiving supply from an agent. The number of the mother calls may be displayed on a ‘bell’ icon 219. The mother may press the ‘bell’ icon 219 and a ‘My Call’ screen 220 will be displayed, for example, on her mobile device display. For example, as illustrated in FIG. 2B the ‘My calls’ screen 220 displays a list of recent mother calls, including for example the following help calls: bedding/towels; help to get out of bed; blood test for the baby. Accordingly, in some cases, next to each call a ‘check’ icon is displayed and colored if the request was taken care and the mother may delete the call if needed. Respectively, the mother's call list is displayed on the staff devices' screens, for example on the mobile phones nurses which are currently on the morning shift at the gynecological ward. For example, as shown in FIG. 2C, the calls list screen 230 includes respectively all the mother calls 220 of FIG. 2B and for each related help call the time of call and the location of the mother 232, i.e. ‘room 112, bed 151121.

Accordingly, a patient in a different department will have other icons included in his graphical interface.

FIG. 2D, FIG. 2E, and FIG. 2F show, respectively, examples of screenshots of a patient and agent interfaces 240, 250 and 260 which automatically match the patient's needs, for example on real-time at the hospital's emergency ward, in accordance with an embodiment. In the example shown in FIG. 2D the graphical interface 240 includes a display that is tailored according to the needs of a patient who was just hospitalized and accordingly his or her graphical interface will include icons that match the patient immediate needs while he or she is for example in intensive care or another ward in the hospital. In some cases, the graphical interface 240 includes the following icons buttons which exactly match the specific needs of the patient: an ‘SOS emergency’ 241—for urgent matters, ‘general help’ 242, ‘supplies’ 243, ‘pain relief’ 244, medical treatment 245. Specifically, by pressing each of these icons a specific help list may be displayed to the patient. For example, once the ‘general help’ 242 is selected a ‘general help’ screen 250 shown in FIG. 2E is displayed to the patient. List 250 may include specific and different needs for each patient at each ward. For example, the patient may select one or more of the following help calls from the list 250: help to get out of bed; not feeling well; disconnect from transfusion; meal after surgery; other. The selected patient's calls may be displayed, for example concurrently, in real-time at the staff devices at the same shift.

According to one embodiment, as illustrated in FIG. 2F the patient may select and send the related staff a specific text message 260 with specific requests or record his message. The patient message 260 may be transmitted and displayed, for example concurrently, in real-time to the staff devices at the same shift, for example to a specific agent who may take care of the request as soon as possible.

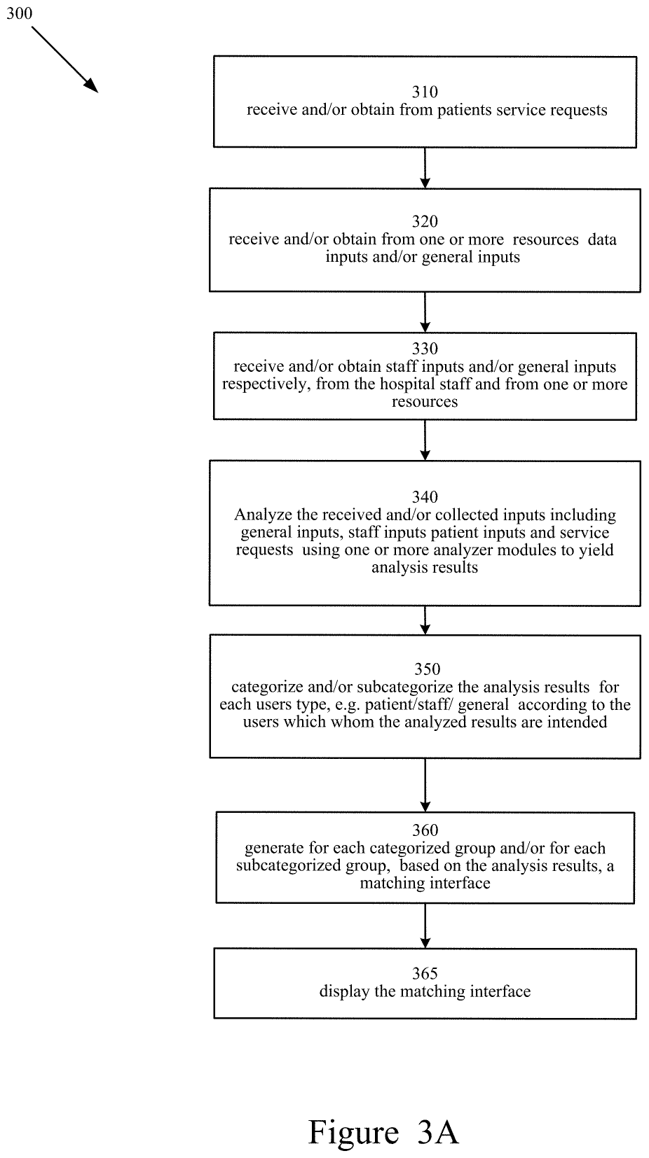

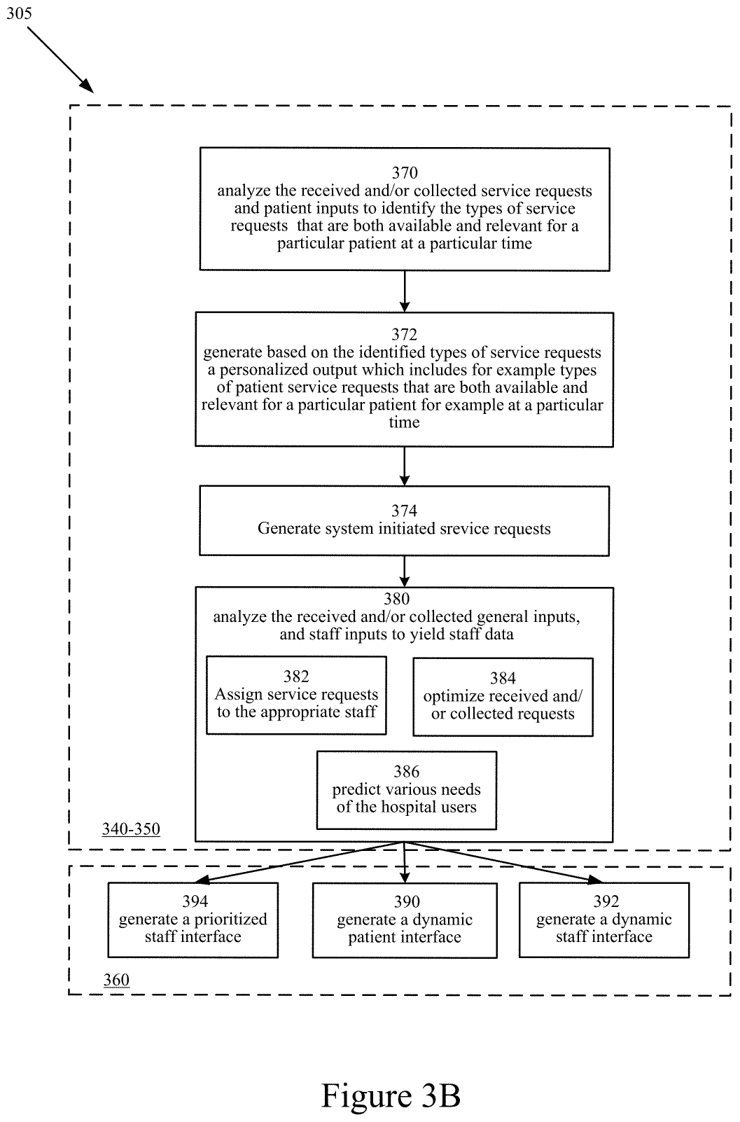

FIG. 3A shows a high-level schematic flowchart of a method 300 for managing requests (e.g. service requests) of users in health institutions (e.g. hospitals) and generating for each user a matching interaction tool such as a matching UI (User Interface) according to the user category (e.g. patient; agent, staff, nurse) to allow effective and optimized operation and control of the requests, in accordance with embodiments. The users may be the hospitals' clientele and/or staff and/or patients who are at home, outdoor, or at the home of the aged. In some cases, the interaction tool may be a UI such as a matching, dynamic and personalized UI for each user to allow the user to effectively and/or optimally operate his mobile application and thus receive the best and efficient medical care and attention. The UI may include for each user one or more screens, pages, and visual elements—like buttons and icons—that enable the user to simply interact with the requested service (e.g. staff such as a nurse, doctor, sanitarian, or any agent). Method 300 may include processing and analyzing inputs and/or service requests received and/or obtained respectively from users and from the hospital's memory and generating a suitable and dynamic UI for each user. The method further includes optimizing health institutions (e.g. hospitals) resources for the benefit of patients, hospital staff and administration, in accordance with embodiments. The optimization includes providing for example optimized patient treatment with respect to time and accuracy. System 100, for example, may be used to implement method 300. However, method 300 may also be implemented by systems having other configurations.

Some stages of method 300 may be carried out at least partially by at least one computer processor, e.g., by processor 26 of the client device and/or system computing subsystem such as processor 24. Respective computer program products may be provided, which comprise a computer-readable storage medium having computer-readable program embodied therewith and configured to carry out of the relevant stages of method 300. In other embodiments, the method includes different or additional steps than those described in conjunction with FIG. 3. Additionally, in various embodiments, steps of the method may be performed in different orders than the order described in conjunction with FIG. 3.

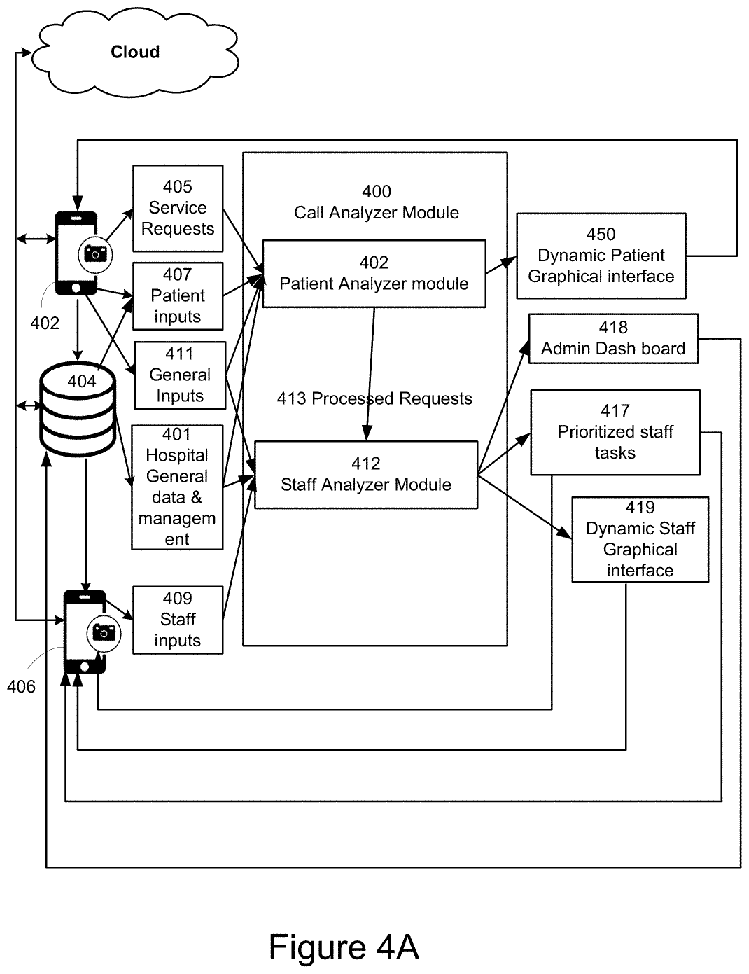

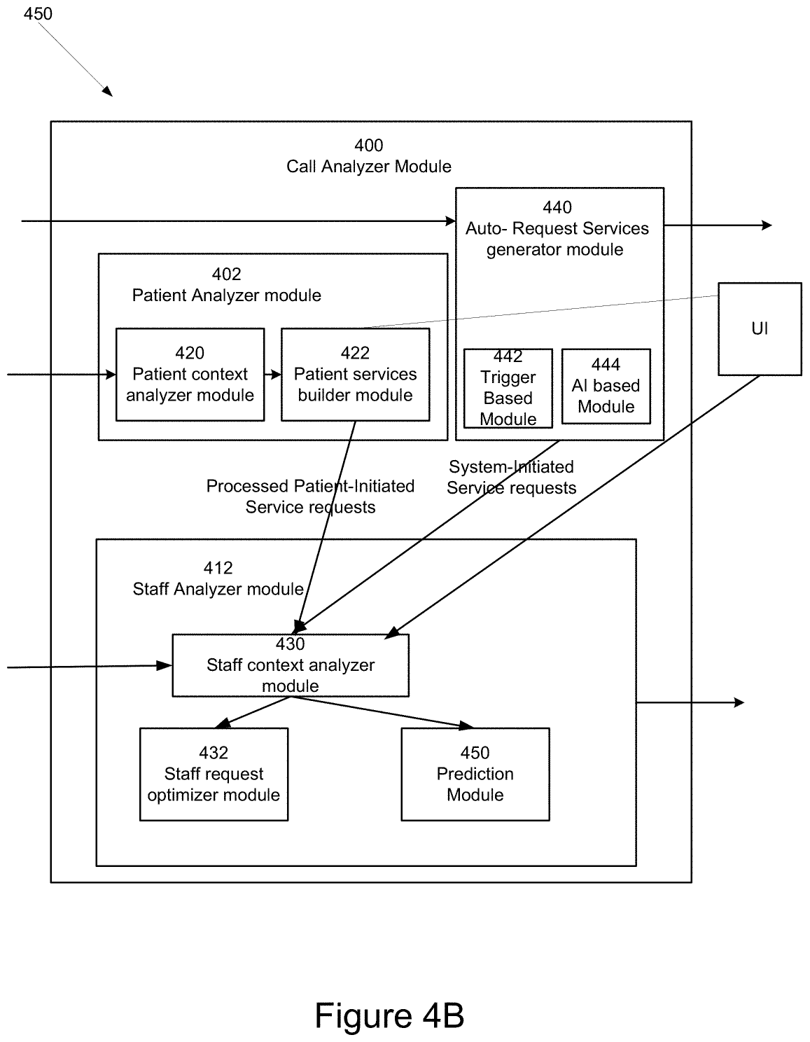

At step 310 service requests are received and/or obtained (for example collected) from patients for example via the network 28 from the patients' devices, such as the patients' mobile phones as illustrated for example in FIG. 1A. In some cases, the service requests are received and/or collected automatically and/or autonomously for example directly from the patient's devices and/or indirectly via a cloud-based storage device such as memory 26 of FIG. 1A. In some cases, the service requests may be uploaded for example to a data storage subsystem such as a cloud-based data storage and later retrieved for example by a call analyzer module 400 as shown in FIG. 4A.

In accordance with embodiments, the service requests may include one or more needs such as specific needs of the patients, for example of a specific patient, which can be fulfilled for example by the staff.

At step 320 patient inputs (e.g. data inputs of patients) and/or general inputs are received and/or obtained manually or automatically from one or more resources. In some cases, the patient inputs may be or may include all relevant information, whether inputted manually into the system or collected via external devices.

In some cases, the resources may be or may include one or more systems and/or devices such as the user's sensors such as cameras or heart-rate sensors, for example, sensors included on the patient's mobile phone. In some cases, the patient inputs include user inputs. In some cases, the patient inputs may be uploaded using the user device such as the user's mobile device (e.g. mobile phone).

In some cases, the patient inputs may include one or more of: demographic details, a condition such as medical condition, when admitted, which department/room/bed, current vitals etc.

In some cases, the patient inputs may be received and/or obtained from the hospital's general data and management systems. In some cases, the patient inputs may be stored at the hospital server 22 for example at the memory 26 (e.g. data storage) shown in FIG. 1A and/or at the Hospital General data & management subsystem 401 which may be in communication with memory 404 shown in FIG. 4A.

In some cases, the general inputs may include data such as one or more of: weather, season, time of day, weekday/weekend/holiday, current hospital occupancy level, etc.

In some cases, the general inputs may include external data such as data received from various external sources including one or more of: system models & algorithms, or other data and AI/other processing, received from external devices and/or from sensors embedded in the user's device; integrated apps; social media and native phone data, IoT devices of the patient smart home devices, or user devices such as the user smart car.

Other external devices may include connected devices (external to phone, like a smart home) test measures, heart rate variability, physiological, endocrinological, neural mechanism Phone data or any data such as native phone data stored at the patient phone.

In some cases, the service requests and/or the patient inputs and/or general inputs may be received from the patient's relatives and/or from authorized sources that were authorized for example from the patient to provide these data (e.g. service requests and/or the patient inputs and/or general inputs).

At step 330 staff inputs are received and/or obtained (for example collected) manually and/or automatically, from the hospital staff and/or from one or more resources such as local or external resources. In some cases, the staff inputs are received or obtained via the network from the staff's devices, for example from the staff's mobile devices (e.g. mobile phones).

In some cases, the staff inputs may include data relating to the staff. For example, the staff inputs may include data comprising one or more of: training, department(s), current availability, current location in the hospital, length of current shift and when it started/finishes, skill level, seniority, etc. In some cases, also general inputs may be received or collected from the staff. In some cases, the staff inputs may include general inputs or patient inputs.

At step 340 the received and/or obtained inputs including general inputs, staff inputs patient inputs and service requests received and collected accordingly from the patients and/or from the staff (e.g. agents) and various systems and devices are analyzed using one or more analyzer modules to yield analysis results

At step 350 the analysis results are categorized according to each users' type, e.g. patients/staff/general such as the hospital database and according to the users which whom the analyzed results are intended. The categorized analysis results also match the needs of each categorized group, and advantageously providing separate, different, and relevant analysis results for each categorized group. Alternatively or additionally, each categorized group may be subcategorized to subgroups according to their status, location, and type, for example, users (e.g. patients or agents) in different wards in the hospital will receive different analysis results according to their role (e.g. nurse/doctor/sanitarian/general database), need (e.g. mother/child/aged/time (day/night), location (e.g. home or at the hospital), medical/health information about the patient, time, etc.

At step 360 for each categorized group and/or for each subcategorized group, based on the analysis results, a matching interaction tool, for example, an interface such as a matching dynamic interface that match the user group category is generated.

For example, in accordance with some embodiments, the users' group type categories include patients/staff (agents)/hospital database, and, respectively, for each group the generated matching interface tool may be presented. The generated interface tool may include the following information (which are based on the analysis results) for each category as follows:

1.For the patient:
A wholly integrated context aware user interface for the patient module, which reflects for each patient which service requests are currently available to him.
2.For the staff (e.g. agent):
A prioritized task list for each agent, including explicit and derived patient requests that he/she is assigned to, and a recommended order of execution
3. For the hospital database:
a. A dashboard with such data as the number of service requests and the time it takes to service a request, broken down by department, request type, agent type, and time related factors (day/night/weekend etc.).
b. Predictions of supply needs and shortages, seasonal personnel needs, hospital bed shortages etc.

Optionally, at step 365 the related interaction tool is displayed on the users' display subsystem, for example on the patients or staff mobile device display or at general information center of the hospital 43 shown in FIG. 1A.

FIG. 3B shows a detailed flowchart 305 of the analysis process of step 340 and further categorization steps 350 and 360 of FIG. 3A, in accordance with embodiments. The analysis process may be operated using an analyzer module such as the call analyzer module 400 illustrated in FIG. 4A and FIG. 4B. The call analyzer module 400 comprises a plurality of modules wherein each module comprises a different purpose and means of operation. In accordance with embodiments, each module contains proprietary software that combines specifically defined traditional algorithms with advanced and novel artificial intelligence for more advanced functions.

It should be noted that, in accordance with some embodiments, the service requests received and collected for example at step 310 are patient-initiated service requests. The patient-initiated service requests may be service requests which are for example expressly requested by the patient using for example his clint device (e.g. smart phone or tablet), as illustrated in FIGS. 2B and 2D. However, the systems devices and method, in accordance with embodiments, are configured and enabled to generate system-initiated service requests. System-initiated service requests may be service requests which may be automatically and/or autonomously deduced and generated for the user (e.g. the patient).

Examples of patient-initiated service requests include sanitarian requests such as requests for toilet/shower help, change of bedding, meal service issues, and/or medical requests such as assistance with pain and the like (shown for example in FIGS. 2B and 2D).

Examples of system-initiated service requests may include scheduled services such as infusions and regular meal services as well as a nurse check up on a patient who has NOT asked for a bedpan all day, or whose oxygenation level (measured by sensors) is too low.

At step 370 the received and/or collected service requests (e.g. patient-initiated service requests), general input and patient inputs are analyzed, using for example a patient analyzer module 402, to identify and/or select the types of service requests the patient that are either one or both available and/or relevant for a particular patient at a particular time. For example, the selected service requests are selected from the service requests which were received from the patients.

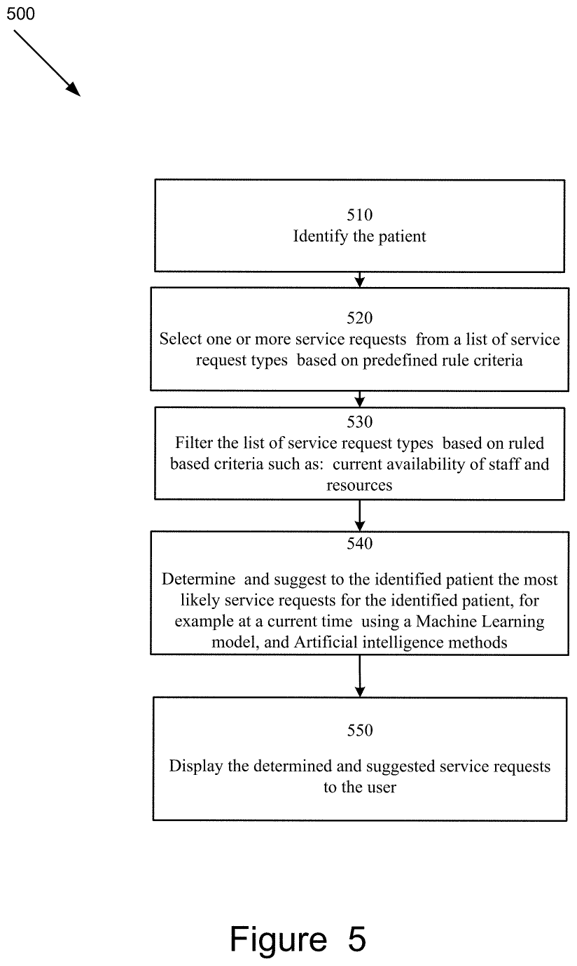

At step 372 based on the identified and/or selected types of service requests (for example which are both available and relevant for a particular patient at a particular time), a personalized output which includes for example types of patient service requests that are both available and relevant for a particular patient for example at a particular time is generated. In other words, the methods and systems, in accordance with embodiments, are configured and enabled to automatically and/or autonomously identify, select and generate patient service requests that specifically match the patients' needs. In some cases, the patient service requests may be produced using a patient services builder module 422. A detailed flowchart of the patient services builder generation is illustrated in FIG. 5.

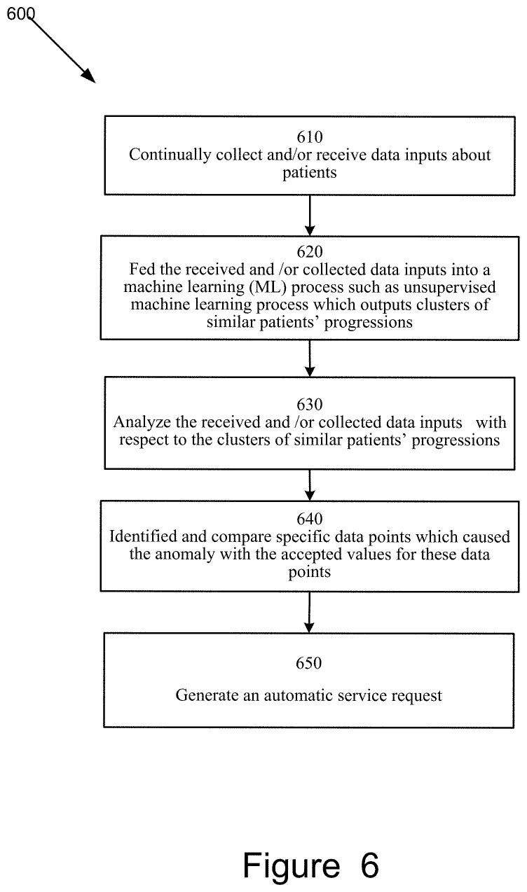

Optionally and/or additionally, at step 374 system-initiated service requests are generated. Specifically, at step 374 for generating system-initiated service requests the received and/or obtained service requests and patient inputs and/or general input are analyzed, using for example the Auto-Request Services Generator Module 440, to automatically and/or autonomously generate service requests for the patients (e.g. system-initiated service requests). Specific details of the generation of the system-initiated service requests are illustrated in FIG. 4B and FIG. 6.

At step 380 the received and/or obtained staff inputs and/or general inputs and/or data received for example from general data and management systems are analyzed, using, for example, a staff analyzer module 412, to yield staff data including for example personalized tasks list specifically for each staff category. Specifically, the analysis of step 380 includes at step 382 assigning service requests to the appropriate staff (e.g. agents—such as nurse or doctor or sanitarian), and at step 384 prioritizing the tasks of each agent, in accordance with embodiments.

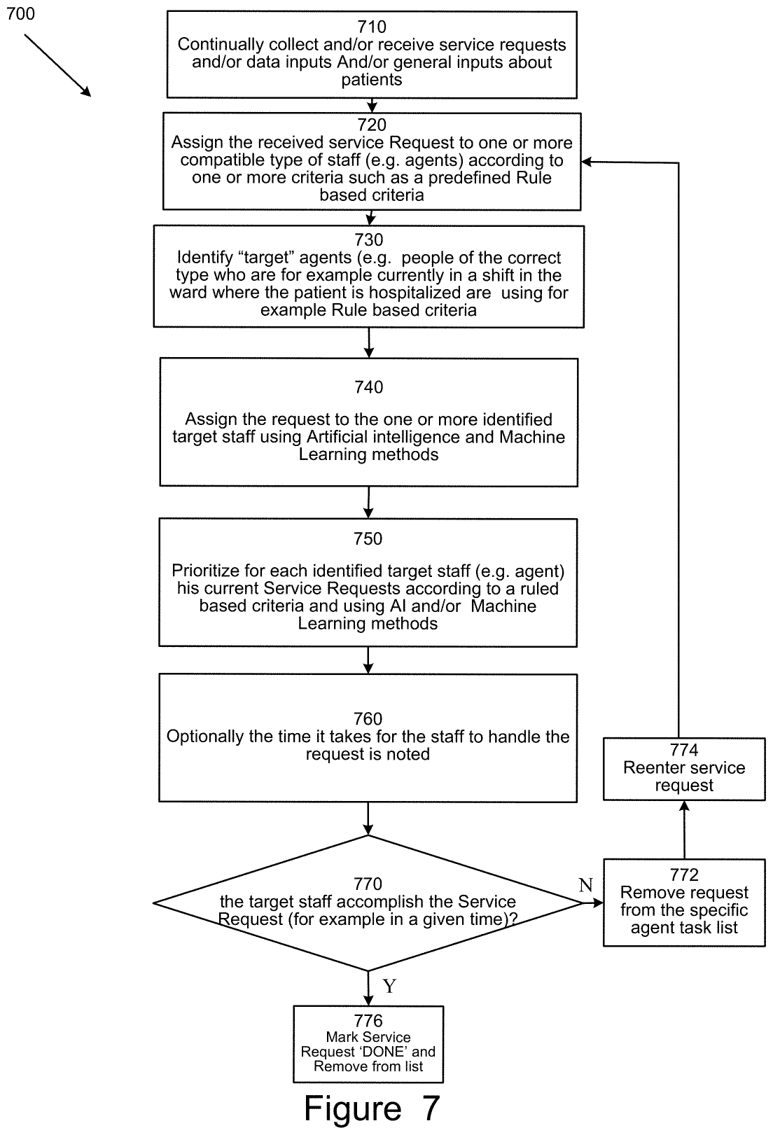

The prioritizing at step 384 includes optimizing received and/or collected requests and assigning service requests to the appropriate staff (e.g. agent) and prioritizing the tasks of each agent, in accordance with embodiments. The optimization method is illustrated in detail in FIG. 7.

In accordance with embodiments, the assigned service requests may be translated to one or more icons on the staff's application user interface which is one of the means by which the agent device interfaces with the call system 100.

Optionally, at step 386 the received and/or collected general inputs, staff inputs, and/or data received for example from general data and management systems are analyzed to predict various needs of the hospital users such as predictions of supply needs and shortages, seasonal personnel needs, hospital bed shortages, etc.

The generation of a matching interface of step 360 of FIG. 3A specifically includes at step 390 generating a dynamic patient interface output such as a graphical interface which is based on the generated service requests for the patients and the patient-initiated service requests and/or the system initiated service requests. In some cases, the output may include a matching patient interface. Specifically, the personalized output comprises a patient interface that is both available and relevant for a particular patient for example at a particular time. More specifically, the output includes graphic symbols such as icons or text on the patient's application user interface which is the primary means by which the patient interfaces with the system (e.g. system 100). In some cases, the output may include various interface modes such as voice messages for the deaf, etc.

At step 392 dynamic staff interface is generated based on the generated staff input. In some cases, the interface may be a dynamic staff Graphical interface which matches the specific staff role (nurse, doctor, sanitarian). At step 394 a prioritized staff tasks list is generated based on the predicted needs.

In accordance with embodiments, all or some steps of FIG. 3A and FIG. 3B may be operated synchronously and continually over time online or offline.

FIG. 4A shows a high-level block diagram of system 100 including a call analyzer module 400, in accordance with embodiments. In some cases, a user may download, for example from a server, an application that includes the call analyzer module 400 and may run the module by his mobile device processor.

The Call Analyzer Module 400 is configured and enabled to process and analyze received and collected inputs and service requests and for example collectively and/or synchronically produce the appropriate and matching result for each user type based on the context of the received and collected inputs and service requests, in accordance with embodiments. In other words, the call analyzer module 400 is configured and enables to yield a suitable output, such as a suitable graphical interface and/or suitable data according to the categorized user type, e.g. patient(s), agent(s), hospital administrator, doctor(s), sanitarian and the like (as described hereinabove with respect to FIGS. 2A-2F and FIGS. 3A-3B).

According to one embodiment, the Call Analyzer Module 400 comprises a patient analyzer module 402 which is configured and enabled to receive data and analyze the data. The data may include service requests 405 uploaded for example using a Dynamic Patient Graphical interface 450 embedded in the patient's device such as patient's mobile phone 402. In some cases, the data may include patient inputs 407 and/or general inputs 411 received or automatically collected for example from a data storage subsystem such as a hospital general data and management system 401 (and memory 404).

The patient analyzer module 402 process and analyze the data to yield the dynamic patient graphical interface 450 which includes, for example, one or more icons which match the patient request. Examples of the dynamic interfaces are shown in FIGS. 2A-2F. In accordance with embodiments, the dynamic patient graphical interface is continually updated over time, therefore if for example the patient moves to a different ward in the hospital or leaves the hospital his graphical interface will automatically be updated according to his updated location and status.

The call analyzer module 400 further comprises a staff analyzer module 412 which is configured to receive and/or collect data including one or more of: staff input 409; and/or general input 411 collected for example from the hospital general data and management subsystem 401; processed patient request 413. The staff analyzer module 412 analyses the data to yield tasks 417 which may be in some cases prioritized at the staff analyzer module and further a dynamic staff Graphical interface 419 which match the specific staff role (nurse, doctor, sanitarian). The dynamic interface may 419 may be displayed for example at the staff's devices, for example in real-time and may be continually updated over time based on the received requests and inputs. In some embodiments, the staff analyzer module 412 analyses the data to yield general data related to the staff and may be displayed at the staff room. The general data may be a dashboard, such as admin dashboard 418 with such data as the number of service requests and the time it takes to service a request, broken down by department, request type, agent type, and time related factors (day/night/weekend etc.). In some cases, the data of the admin dashboard 418 may be stored at memory 404.

FIG. 4B is a block diagram 450 illustrating in detail an example of the modules included in the call analyzer module 400. The call analyzer module 400 comprises a plurality of modules wherein each module comprises a different purpose and means of operation. In accordance with embodiments, each module contains proprietary software that combines specifically defined traditional algorithms with advanced artificial intelligence for more advanced functions.

According to one embodiment, the call analyzer module 400 comprises a patient context analyzer module 420, a patient services builder module 422, a staff context analyzer module 430 a staff request optimizer module 432, a predictions module 450 and an Auto request services generator module 440.

According to some embodiments, the patient context analyzer module 420, the patient services builder module 422, may be included in a patient analyzer module 402, while the staff context analyzer module 430 and the staff request optimizer module 432 and the predictions module 450 are included in a staff analyzer module 412. It is noted, however, the other configurations of the modules may be provided in accordance with embodiments.

Patient Analyzer Module 402

Generally, the services most relevant to any specific patient are determined by for example two factors: A) what is possible and B) what is likely. A) What is possible: not all services are relevant to all patients, for example, lactation consolation is only relevant for women in the maternity ward. Moreover, not all services are relevant at all times: For example, there is no sense in offering a change of bedding if the ward ran out of clean laundry. B) What is likely: although many services are technically available to many patients, the likelihood of requesting them is very variable. For example, a newly diagnosed oncology patient is more likely to ask for a social worker than a teenager who broke his leg.

In accordance with embodiments, the patient context analyzer module 420 is configured and enabled to receive or collect data and analyze data to identify the types of service requests by the patient that are both available and relevant for a particular patient at a particular time and transmit the identified service requests that are both available and relevant for a particular patient to the patient services builder module 422. Generally, the received data may include any data that is relevant to the patient and exist in the system 100 for example in the memory 26 or the system central data storage 21. Specifically, the data may include service requests 405 and/or patient inputs 407 and/or general inputs 411 received for example using the patient device or retrieved from the hospital's data storages (e.g. memory 404 of FIG. 4A or memory 26 of FIG. 1A) or from any external resources.

Based on the identified type of services a personalized output is generated by the patient services builder module 422. The personalized output may include a matching patient interaction tool such as an interface. Specifically, the personalized output comprises a patient interface (e.g. UI) that is both available and relevant for a particular patient for example at a particular time. More specifically, the output includes graphic symbols such as icons or text on the patient's application user interface which is the primary means by which the patient interfaces with the system (e.g. system 100). In some cases, the output may include various interface modes such as voice messages for the blind, simplified presentation for people with cognitive issues, right colors for color-blind people, etc.

In accordance with embodiments, a method 500 for identifying the types of service requests that are both available and relevant for a particular patient at a particular time as operated for example by the patient services builder module 422 is illustrated in FIG. 5.

The Auto-Request Services Generator Module 440 is configured and enabled to automatically and/or autonomously generate service requests for users (e.g. patients). The need to automatically and/or autonomously generate service requests for users such as patients according to their real need and in real-time is especially critical in a hospital setting, where patients might need attention, but they are unaware of the need or unable to request for example due to extreme workload on the medical staff as a result of crises such as a pandemic or multi-casualty incident.

A specific example of the efficiency and importance of Anomaly Detection as operated using the Auto-request services generator module 440 may relate to a childbirth process. Following childbirth, women are instructed to call a nurse the first time they get up to use the bathroom. The actual time this happens varies greatly from one woman to the next, since the circumstances of each birth differ. Normally, the woman will call for the nurse on her own when she requires this assistance. However, if she doesn't, the Auto-request Services generator module will automatically and autonomously notice an anomaly compared to other women, and the nurse will be alerted to pop in and offer to escort the patient, thus avoiding a potential fall. For example, the Auto-request Services generator module 440 will automatically and autonomously press the Urgent button in the mother UI shown in FIG. 2A and the nurse will receive the specific task in her Action Items list at her UI as shown in FIG. 2C.

According to one embodiment, the Auto-Request Services Generator Module 440 includes two types of automatic request generation modules: trigger based automatic service requests (ASR) module 442, and AI (Artificial Intelligence) based ASR module 444. The Trigger-based automatic service requests (ASR) module 442 generates service requests using one or more specific rules (e.g. triggers) that may be defined for example by the hospital or ward. For example, the rules may include the following triggers: every patient needs a change of bedding every day; Or—any patient who requested pain medication must be re-examined 3 hours later. In general, the Trigger-based ASRs are configured and enabled to cover common, broad cases.

The AI based ASR module 444 uses Artificial Intelligence methods to recognize and flag situations such as patient medical situations that might require attention and/or treatment. For example, surgical patients normally experience pain 2-4 hours after anesthesia wears off, and request pain killers. In the event that a request for pain killers was not made by the patient, module 444 will automatically generate a request for example in real-time in the name of the patient that must receive the pain killer as soon as possible.

According to one embodiment, the AI based ASR module 444 use Anomaly Detection methods based on AI and/or ML methods to generate automatic service requests. Specifically, Anomaly Detection methods include identifying data points or ranges that deviate from a dataset's normal behavior. More specifically, in accordance with embodiments, the AI based ASR module 444 uses Anomaly Detection method to identify when the course of a specific patient's hospitalization deviates significantly from what is expected for him. In some cases, the Anomaly Detection method includes algorithms using unsupervised k-means clustering and modified k-means algorithms

In accordance with embodiments, the staff context analyzer module 430 is configured and enabled to receive data and analyze the data to identify the types of service requests by the patient that are both available and relevant for a particular patient or staff at a particular time and transmit the identified service requests that are both available and relevant for a particular patient and staff to the staff request optimizer module 432 and/or to the prediction module 450. At the staff context analyzer module 430 one or more algorithms are configured to identify exactly a long list of dozens of data points, then run experiments and iterate to get best results.

The Staff Request Optimizer Module 432 is configured and enabled to assign service requests to the appropriate staff (e.g. agents such as nurses or doctors), and to prioritize the tasks of each agent, in accordance with embodiments. The assigned service requests may be translated to one or more icons on the staff's application user interface 419 which is one of the means by which the agent device interfaces with the call system 100.

According to another embodiment, the Staff Request Optimizer module 432 may be also configured to move requests (e.g. service requests) from one agent to another, for example by sending the requests to another staff member according to a ruled based criteria stored for example at the memory 26 of FIG. 1 or memory 404 of FIG. 4A.

In operation, service requests may be uploaded by the user (e.g. patient) via the network using for example the user device (e.g. the patient's mobile phone) and received (or first processed and then received) for example at a staff context analyzer module 430 using one of two ways: a) directly requested by a patient, or b) as ASRs from the Auto request generator module 440. In accordance with embodies, the Staff Request Optimizer (SRO) 432 deals with the Service Requests as they enter the system. Once received the Staff Request Optimizer (SRO) 432 performs two tasks: 1) prioritizing service requests and 2) assigning the service requests to the staff (e.g. to agents).

FIG. 5 shows a flowchart 500 of a method for identifying the types of service requests that are both available and relevant for a particular patient for example at a particular time, in accordance with embodiments.

Some stages of method 500 may be carried out at least partially by at least one computer processor, e.g., by processor 36 of the client device and/or system computing subsystem such as processor 24. In accordance with embodiments method 500 may be carried out using the patient services builder module 422. Respective computer program products may be provided, which comprise a computer-readable storage medium having computer-readable program embodied therewith and configured to carry out of the relevant stages of method 500. In other embodiments, the method includes different or additional steps than those described in conjunction with FIG. 5. Additionally, in various embodiments, steps of the method may be performed in different orders than the order described in conjunction with FIG. 5.

At step 510 the patient is identified, using for example one or more known identification methods such as ID number, phone number, IRS (Interface Requirements Specification). The patient identification includes for example an identification field or several fields that the patient may fill using for example his mobile phone.

At step 520 one or more service requests are selected from a list of service request types available for example in the system 100 (e.g. at the client device App) based on predefined rule criteria. Specifically, the selected service requests are selected based on the service request types that apply to the current patient based on predefined criteria such as ward and condition relevant to the patient.

At step 530 the list of service request types is filtered based on ruled based criteria such as: current availability of staff and/or resources (each ward will be able to determine its own cutoffs) and the like.

At step 540 the most likely service requests for the identified patient, for example at a current time (as explained in details hereinbelow) are determined and suggested to the identified patient using one or more Machine Learning model and/or Artificial intelligence methods such as modified collaborative filtering algorithms incorporating transient data.

According to one embodiment, a Time-Based Collaborative Filtering method is used to determine which service requests to suggest for a particular patient at a particular time. Specifically, the Collaborative Filtering method uses similarities between users and items, for example simultaneously, to provide recommendations to the user (e.g. the patient). The Collaborative Filtering method includes receiving data about the users (e.g. patients) and Data about the items (e.g. requests). Additionally, the Time-Based Collaborative Filtering incorporates in its training data (in addition to user data (e.g. patient data such as patient inputs) and item data (such as general data)) also transient data including one or more of: Time of day, day of week, holiday yes/no, season, etc. The received data is processed and analyzed (e.g. trained) using one or more machine learning and AI methods to yield recommendations to the user (e.g. the patient). Advantageously, while most recommender systems recommend items to users independent of time, the Patient Services Builder may be a Time Aware recommendation engine incorporating the ability to recommend the items most appropriate for the user at the current time.

In accordance with embodiments the user data, item data and transient data may include one or more of the following specific data:

Transient data: Time of day, day of week, holiday yes/no, season etc.

Data about the users (e.g. patients) may include: ward, age, gender, level of mobility, days/hours since admission, number of previous requests (by category) this patient made etc.

Data about the items (e.g. requests) may include: type (medical/service/consultation), popularity etc.

At step 550 the determined and suggested service requests are displayed to the user (e.g. patient) using an interaction tool such as a graphical interface which presents to the patient the appropriate and relevant service requests using text and/or icons as shown for example in FIGS. 2A-2F.

FIG. 6 shows a flowchart 600 of a method for automatically and/or autonomously generating service requests that are both available and relevant for a particular patient for example at a particular time, in accordance with embodiments.

Some stages of method 600 may be carried out at least partially by at least one computer processor, e.g., by processor 36 of the client device and/or system computing subsystem such as processor 24. In accordance with embodiments method 600 may be carried out using the Auto Request Services generator module 440. Respective computer program products may be provided, which comprise a computer-readable storage medium having computer-readable program embodied therewith and configured to carry out of the relevant stages of method 600. In other embodiments, the method includes different or additional steps than those described in conjunction with FIG. 6. Additionally, in various embodiments, steps of the method may be performed in different orders than the order described in conjunction with FIG. 6.

At step 610 data inputs about patients (e.g. all patient such as patient inputs 407 or service requests 405 from all patients in the hospital) are continually collected and/or received over time. The inputs may include for example one or more of: vitals, responses and/or requests for services. In some cases, the inputs are collected in real-time.

At step 620 the received and/or collected data inputs are fed into a machine learning (ML) process such as an unsupervised machine learning process which outputs clusters of similar patients' progressions.

At step 630 the received and/or collected data inputs are analyzed with respect to the clusters of similar patients' progressions. The analysis may include comparing the received and/or collected data inputs to the clusters of similar patients' progressions. Accordingly, patients whose data is within the cluster boundaries are considered as having “accepted” progressions, while any patient whose hospitalization profile continually or suddenly appears ‘far’ from any of the clusters is flagged. The term ‘far’ may be defined as outside of the cluster boarders. For example, if the data is represented as points in an n-dimensional space, a cluster is a group of such points that all fall within a certain area in said space. Once the clusters are defined, their boarders are calculated. Data points that are not within cluster boarders are anomalities.

At step 640 specific data points that caused the anomaly are identified and compared with the accepted values for these data points.

At step 650 an automatic service request is generated based on the comparison and includes an accurate service request including a specific need of the patient, which should be fulfilled for example by an agent.

FIG. 7 shows a flowchart 700 of a method for automatically and/or autonomously optimizing received and/or collected requests including assigning service requests to the appropriate agent, and prioritizing the tasks of each agent, in accordance with embodiments.

Some stages of method 700 may be carried out at least partially by at least one computer processor, e.g., by processor 36 of the client device and/or system computing subsystem such as processor 24. In accordance with embodiments method 700 may be carried out using the Staff Request Optimizer module 432. Respective computer program products may be provided, which comprise a computer-readable storage medium having computer-readable program embodied therewith and configured to carry out of the relevant stages of method 700. In other embodiments, the method includes different or additional steps than those described in conjunction with FIG. 7. Additionally, in various embodiments, steps of the method may be performed in different orders than the order described in conjunction with Figure.

At step 710 service requests (e.g. of all patients such as service requests 405) and/or inputs (e.g. patient inputs 407) and/or general inputs are continually collected and/or received over time. In some cases, the service Request are collected in real-time. In some cases, the service Request is collected offline.

At step 720 the received service Request are Assigned to one or more compatible type of staff (e.g. agents) according to one or more criteria such as a predefined Rule based criteria. In some cases, the criteria include specifications such as which type of staff is required as some requests can only be fulfilled by nurses, others only by kitchen staff etc.

At step 730 “target” staff, such as agents (e.g. people of the correct type who are for example currently in a shift in the ward where the patient is hospitalized and have the capabilities to best answer the patient's need) are identified using, for example, Rule based criteria.

At step 740 the request to the one or more identified target agents is assigned using Artificial intelligence and Machine Learning methods.

In some embodiments, the service requests (of step 740) are assigned to one of the currently available agents using a Logistic Regression method. The Logistic Regression method, in accordance with embodiments, includes characterizing each request (e.g. service request) by one or more attributes such as how often this request is made, how fast its handled, on average, how urgent it is, etc. The available agents are also categorized for example based on their attributes such as how long they have been in the shift, how many requests they have on their current list and their seniority. The method then uses a variant of for example Multinomial logistic regression to assign the service request to the agent best suited to handle it.

Using Logistic Regression to Assign Service Requests to Agents:

In accordance with embodiments, method 700 includes assigning the service requests to one of the currently available agents as illustrated in step 740. In accordance with one embodiment, each request is characterized by many attributes such as how often this request is made, how fast its handled, on average, how urgent it is, etc. The available agents are also categorized based on their attributes such as how long they have been in the shift, how many requests they have on their current list and their seniority. The system then uses a variant of Multinomial logistic regression to assign the service request to the agent best suited to handle it.

At step 750 for each identified target staff (e.g. agent) his current Service Requests are prioritized according to a ruled-based criteria, using AI and/or Machine Learning methods.

In some embodiments, the service requests prioritization of step 750 is performed using Random Forest methods. Specifically, in operation at any given time, each agent has multiple service requests, which are presented to him for the method to automatically prioritize the service requests based on ruled based criteria. The prioritization may be based on one or more of the following rules: urgency, how long the service request has been in the queue, how much time it normally takes to handle this request, and how “needy” the patient is. The method, in accordance with embodiments, includes using the supervised machine learning algorithm such as “Random Forest” modified by “feature importance” to an ordered list for each agent. For example, the method includes determining which of several service requests assigned to a certain agent is of highest priority. The training data will consist of dozens of data points about the SR, and a humanly tagged categorical indication of priority. This will be repeated to generate an ordered list

Other algorithms may be used.

Optionally, at step 760 the time it takes for the target agent to handle the request is measured and noted, in accordance with embodiments.

Optionally, at step 770 a binary decision can be made regarding whether the target agent of the staff accomplished the Service Request for example in a given time. For example, in some cases, if the agent fails to complete a Service Request after a given time, then at step 772 it will be removed from the agent's task list and reentered into the general task list at step 774. If the agent accomplished the Service Request it will be deleted at step 776 from a general list of service requests and/or marked as ‘DONE’.

Using Random Forest to Prioritize the Service Requests:

At any given time, each agent has multiple service requests, which are presented to him in order of priority. The priority is based on urgency, as well as how long the service request has been in the queue, how much time it normally takes to handle this request, and how “needy” the patient is. The system uses the supervised machine learning algorithm called “Random Forest” modified by “feature importance” to an ordered list for each agent.

### Authentication and Encryption Methods

In accordance with another embodiment, the systems, devices and methods are configured and enabled to allow writing and reading of secure messages on a patient chart (or similar) to let the medical staff such as nurses and doctors leave notes for each other, for example, when the patient last ate, what they ate, last medication time, dosage, shower, bathroom use, etc. Entry can be written or in some cases (like food eaten) entered via a picture.

It is often the case that different doctors and different nurses have varying ideas on what the patient needs, when they need it, and how something should be done. The patient and the family are left to try and sort it out.

Advantageously, in accordance with embodiments, a record of the different views may be stored at data storage such as the cloud-based data storage and may be available for review and discussion using one or more authentications methods.

In one embodiment, access to messages and the patient's chart may be protected on the basis of for example RFID methods and/or other signal technology (e.g. Bluetooth signal from a phone) in all staff tags for the ward or any specified doctor.

In accordance with other embodiments, the systems, devices and methods may include different graphical interface views available for doctors, nurses, patients based on ID, or fingerprint, or a special pen, or facial recognition, vocal recognition, password, etc., presented for example as symbols (icons) on the screen as illustrated in FIGS. 2A-2F.

An alternative or additional method for authentication for accessing patient data includes using one or more sensors such as cameras available on the user's phone and initiate one or more recognition methods such as facial recognition on the: 1. Doctor and 2. On the patient or on some recognizable sign at the patient's bedside, e.g. QR code or hospital property ID.

In some cases, the authentication process may be categorized according to one or more authentication levels according to the type of data for example when a doctor is authenticated for a patient's data he can access all relevant data, alternatively for data that can only be shared with the patient's agreement, each participant i.e. doctor/patient, receives part (e.g. half) the data and requires the 2nd half to see the full picture.

In accordance with some embodiments, for 2D presentations the medical data might require putting two screens near each other, or for both phones to project onto a common screen. With VR and possible holographic models both participants' data would be needed to complete the hologram. In some embodiments, the data may be divided, for example in half, and each party controls half the data or the data could be encrypted with each part holding either half of the decryption key or in a double encryption system each party would hold one key out of the two required for decryption.

In another embodiments, the system and methods may include two devices for authentication process. For example, with the use of two phones and four cameras for doctor and patient (two cameras at each phone), there can be a full two way authentication by both doctor and patient in order to share information, or both would need to authenticate to a third party or device, like a common screen, in order to share data with the device or person.

In some embodiments, when the patient is checked into or out of different departments or services, module 400 is updated from that department.

In some cases, data analysis provided by module 400 may compare the patients' current state and activities, and prepare all parties for the next steps, with notes added to the patient's profile. For example, if the patient has done x-rays and there are not yet results, the system will note that Mill may be necessary depending on results.

When results are delivered the module 400 may suggest possible scenarios for use by nursing and medical staff for preparation of the ward, room, OR, etc., advantageously, making the logistics and psychological adjustments more efficient. Generally, module 400 is configured and enabled to give predictive direction based on the big data analysis of similar cases and their development.

Data analysis may provide a basic path for what the patient will be doing, experiencing and requesting and at least a general timeline that can be used by the staff and patients to prepare for upcoming stages in treatment or recovery. For example, what do people who have undergone a certain procedure generally ask for? And when? Nurses should have the requisite medication, devices, contact information, etc. available in advance of the need.

In further embodiments, the processing unit may be a digital processing device including one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random-access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, the system disclosed herein includes one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device.

In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media. In some embodiments, the system disclosed herein includes at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, the system disclosed herein includes software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

In some embodiments, the system disclosed herein includes one or more databases, or use of the same. In view of the disclosure provided herein, those of skill in the art will recognize that many databases are suitable for storage and retrieval of information as described herein. In various embodiments, suitable databases include, by way of non-limiting examples, relational databases, non-relational databases, object-oriented databases, object databases, entity-relationship model databases, associative databases, and XML, databases. In some embodiments, a database is internet-based. In further embodiments, a database is web-based. In still further embodiments, a database is cloud computing-based. In other embodiments, a database is based on one or more local computer storage devices.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of that additional elements.

It is to be understood that where the claims or specification refer to “a” or “an” element, such reference is not be construed that there is only one of that elements. It is to be understood that where the specification states that a component, feature, structure, or characteristic “may”, “might”, “can” or “could” be included, that particular component, feature, structure, or characteristic is not required to be included. Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described. Methods of the present invention may be implemented by performing or completing manually, automatically, or a combination thereof, selected steps or tasks.

The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. The present invention may be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for managing service requests of patients and staff in a hospital and generating, respectively, for each of said patients and staff a matching dynamic interface, the method comprising:
receiving or obtaining via the network from the patient' devices the service requests;
receiving or obtaining, patient inputs and general inputs from one or more resources;
receiving or obtaining staff inputs respectively from the staff or from the one or more resources;
analyzing the received and obtained service requests, data inputs, staff inputs and general inputs using one or more analyzer modules to yield analysis results;
categorizing the analysis results to groups categories wherein said categorized groups comprise staff groups, patient groups and hospital database;
generating for each of said categorized groups the matching dynamic interface based on the analysis results, wherein the matching dynamic interface comprises output data which match the group category medical needs and whom the analysis results are intended.

2. The method of claim 1 wherein the analysis comprises:
analyzing the received and obtained service requests and data inputs using a patient analyzer module; and
identifying types of service requests in said obtained service requests that are both available and relevant for a particular patient of said patients at a particular time;
displaying said identified service requests at said matching dynamic interface.

3. The method of claim 1 wherein the analysis comprises:
analyzing the received and obtained service requests and data inputs using an Auto-Request Services Generator Module;
automatically or autonomously generating system-initiated service requests for the patients.

4. The method of claim 3 wherein the system-initiated service requests for the patients are generated using one or more specific rules or using Artificial Intelligence (AI) methods to recognize and flag situations.

5. The method of claim 4 wherein said AI methods are Anomaly Detection methods.

6. The method of claim 1 wherein the analysis comprises:
analyzing the received and collected general inputs and staff inputs or data received from general data and management systems using a staff analyzer module to yield staff data.

7. The method of claim 6 wherein the staff data comprises personalized tasks list specifically for each staff category.

8. The method of claim 6 comprising:
assigning service requests to the appropriate staff.

9. The method of claim 7 comprising:
prioritizing tasks in the personalized tasks list.

10. The method of claim 7 comprising:
automatically or autonomously optimizing the received and obtained requests and assigning service requests to the appropriate agent

11. The method of claim 7, comprising:
displaying the prioritized tasks on the staff interface.

12. The method of claim 1 comprising:
generating a separate dynamic interface for the staff groups and for the patient groups.

13. The method of claim 1 comprising:
analyzing the received and obtained collected general inputs, staff inputs or data received for example from general data and management systems;
predicting service request needs of the patients.

14. The method of claim 13 wherein the service request needs comprise one or more of:
supply needs and shortages, seasonal personnel needs, hospital bed shortages.

15. The method of claim 1 wherein the service requests comprise any specific need of the patients, which can be fulfilled by the staff.

16. The method of claim 1 wherein the staff inputs are received or obtained via the network from the staff's devices.

17. The method of claim 1 wherein the matching dynamic interface is a dynamic graphical interface comprising the output data, and wherein said output data is configured and enabled to continually, automatically or autonomously being updated and changed according to the patients' needs, status and location.

18. The method of claim 2 wherein said output data comprises one or more of:
graphical data, one or more icons.

19. A system for managing service requests of patients and staff in a hospital and generating, respectively, for each of said patients and staff a matching and dynamic interface, the system comprising:
a memory, which is configured to hold one or more of patient inputs or general inputs; and
a processor, said processor is configured to:
receive or obtain via the network from the patient' devices service requests;
receive or obtain, the patient inputs and general inputs from the memory;
receive or obtain staff inputs respectively from the staff or from the one or more resources;
analyze the received and obtained service requests, data inputs, staff inputs and general inputs using one or more analyzer modules to yield analysis results;
categorize the analysis results to groups categories wherein said categorized groups comprise staff groups, patient groups and hospital database;
generate for each of said categorized groups the matching dynamic interface based on the analysis results, wherein the matching dynamic interface comprises output data which match the group category medical needs and whom the analysis results are intended.

20. A computer software product, comprising a non-transitory computer-readable medium in which program instructions are stored, which instructions, when read by a computer, cause the computer to provide a method for managing service requests of patients and staff in a hospital and generating for each of said patients and staff a matching dynamic interface, the method comprising:
receiving or obtaining via the network from the patient' devices service requests;
receiving or obtaining, patient inputs and general inputs from one or more resources;
receiving or obtaining staff inputs respectively from the staff or from the one or more resources;
analyzing the received and obtained service requests, data inputs, staff inputs and general inputs using one or more analyzer modules to yield analysis results;
categorizing the analysis results to groups categories wherein said categorized groups comprise staff groups, patient groups and hospital database;
generating for each of said categorized groups the matching dynamic interface based on the analysis results, wherein the matching dynamic interface comprises output data which match the group category medical needs and whom the analysis results are intended.
